(19) United States
(12) Patent Application Publication
(10) Pub. No.: US 2025/0064409 A1
(43) Pub. Date: Feb. 27, 2025

(54) X-RAY CT APPARATUS

(71) Applicant: FUJIFILM Corporation, Tokyo (JP)

(72) Inventors: Hiroki SADAMARU, Chiba (JP); Masanori KAKIMOTO, Chiba (JP)

(73) Assignee: FUJIFILM Corporation, Tokyo (JP)

(21) Appl. No.: 18/808,061

(22) Filed: Aug. 18, 2024

(30) Foreign Application Priority Data
Aug. 23, 2023 (JP) 2023-135244

(51) Int. Cl. A61B 6/03; A61B 6/00; A61B 6/04

(52) U.S. Cl. CPC A61B 6/035; A61B 6/4435; A61B 6/0407

(57) ABSTRACT

An object is to provide an X-ray CT apparatus capable of improving workability of maintenance. An X-ray CT apparatus includes a plurality of components including an X-ray source and an X-ray detector; a rotation frame on which the plurality of components are mounted and which rotates; and a support frame that rotatably supports the rotation frame, in which the support frame includes one or more lock mechanisms including a fixing shaft and a support member that movably supports the fixing shaft, the support frame includes one or more lock fixing units engageable with the fixing shaft, and the lock mechanism switches between a locked state in which rotation of the rotation frame is restricted and an unlocked state in which the rotation of the rotation frame is allowed.

### CROSS-REFERENCE TO RELATED APPLICATION

The present application claims priority under 35 U.S.C § 119 (a) to Japanese Patent Application No. 2023-135244 filed on Aug. 23, 2023, which is hereby expressly incorporated by reference, in its entirety, into the present application.

### BACKGROUND OF THE INVENTION

### 1. Field of the Invention

The present invention relates to an X-ray CT apparatus, and particularly relates to an X-ray CT apparatus in which maintenance work is easily performed.

### 2. Description of the Related Art

The X-ray CT apparatus includes a rotation frame on which an X-ray tube, an X-ray detection unit, and the like are mounted, and a support frame that rotatably supports the rotation frame. The rotation frame is rotationally driven by a rotation belt driven by a motor. The rotation frame, the rotation belt, and the like are accommodated in a housing (a gantry cover). The stop of the rotation frame during the operation is performed by stopping the rotation belt using a brake device.

In a case where the X-ray CT apparatus is maintained, the gantry cover is removed. The rotation frame is locked so that the rotation of the rotation frame is restricted. In a locked state, a component mounted on the X-ray CT apparatus is repaired or replaced. JP2011-147652A discloses a mechanism for locking a rotation frame. JP2011-147652A discloses that a tip part of a lock mechanism provided in a support frame engages with a fixing hole or a fixing protrusion provided in the rotation frame to lock the rotation frame.

### SUMMARY OF THE INVENTION

However, in a case where the rotation frame is locked, an operator needs to cause the lock mechanism and the fixing hole or the like, which are located at a deep position, to engage with each other. Therefore, this has been a cause of a decrease in workability of maintenance.

The present invention has been made in order to solve the above-described problems, and an object thereof is to provide an X-ray CT apparatus capable of improving workability of maintenance.

An X-ray CT apparatus according to a first aspect includes a plurality of components including an X-ray source and an X-ray detector; a rotation frame on which the plurality of components are mounted and which rotates; and a support frame that rotatably supports the rotation frame, in which the support frame includes one or more lock mechanisms including a fixing shaft and a support member that movably supports the fixing shaft, the support frame includes one or more lock fixing units engageable with the fixing shaft, and the lock mechanism switches between a locked state in which rotation of the rotation frame is restricted and an unlocked state in which the rotation of the rotation frame is allowed.

In an X-ray CT apparatus according to a second aspect, in the locked state in which the fixing shaft of the lock mechanism and the lock fixing unit engage with each other, any one of the plurality of components is positioned at a position below a rotation axis of the rotation frame.

In an X-ray CT apparatus according to a third aspect, any one of the plurality of components is the X-ray source.

In an X-ray CT apparatus according to a fourth aspect, the one or more lock fixing units include a plurality of lock fixing units.

In an X-ray CT apparatus according to a fifth aspect, any component of the plurality of components is positioned at a predetermined position depending on a combination of locked states in which one of the plurality of lock fixing units and the fixing shaft of the lock mechanism engage with each other.

In an X-ray CT apparatus according to a sixth aspect, the one or more lock mechanisms include a plurality of lock mechanisms.

In an X-ray CT apparatus according to a seventh aspect, any component of the plurality of components is positioned at a predetermined position depending on a combination of locked states in which the fixing shaft of one lock mechanism of the plurality of lock mechanisms and the lock fixing unit engage with each other.

In an X-ray CT apparatus according to an eighth aspect, the fixing shaft is disposed to be offset from the support member.

In an X-ray CT apparatus according to a ninth aspect, the lock mechanism includes an indicator indicating whether the support member is at a position where the rotation frame is locked or at a position where the rotation frame is unlocked.

In an X-ray CT apparatus according to a tenth aspect, the lock mechanism includes an elastic member that biases the support member to a position where the rotation frame is in the unlocked state.

An X-ray CT apparatus according to an eleventh aspect further includes a motor that rotates the rotation frame; and a processor that turns off power of the motor in a case where the rotation frame is in the locked state.

In an X-ray CT apparatus according to a twelfth aspect, in a state where the fixing shaft engages with the lock fixing unit, the fixing shaft maintains the rotation frame in the locked state against a maximum output of the motor.

According to the present invention, workability of maintenance can be improved.

### BRIEF DESCRIPTION OF THE DRAWINGS

FIG. 1 is a perspective view describing an appearance of an entire X-ray CT apparatus.

FIG. 2 is a block diagram illustrating an overall structure of an X-ray CT apparatus according to an embodiment.

FIGS. 3A to 3C are external views of a gantry cover.

FIG. 4 is a front view of a turn plate of the X-ray CT apparatus of the embodiment with the gantry cover removed.

FIG. 5 is a front view of a state where a rotation frame is detached from the turn plate of FIG. 4.

FIG. 6 is a diagram for describing a rotation frame in a locked state.

FIG. 7 is a diagram for describing a lock mechanism in a locked state.

FIG. 8 is a diagram for describing a lock mechanism in an unlocked state.

FIGS. 9A and 9B are diagrams for describing an indicator of the embodiment.

### DESCRIPTION OF THE PREFERRED EMBODIMENTS

An X-ray computed tomography (CT) apparatus according to an embodiment of the present invention will be described.

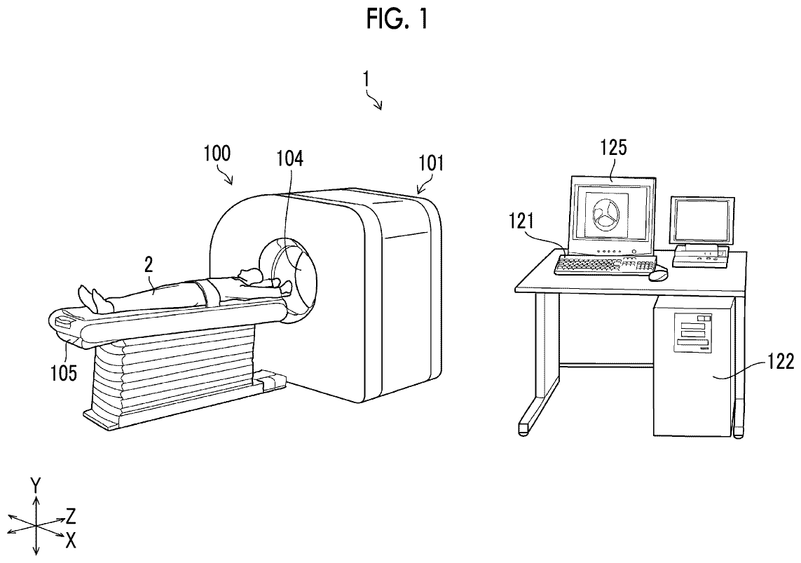

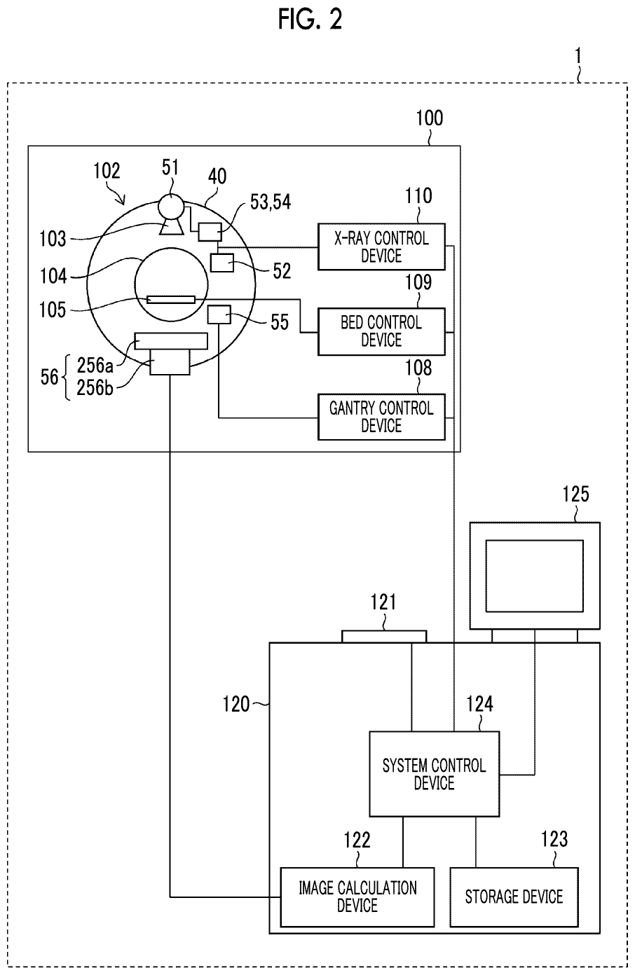

First, an overall structure of the X-ray CT apparatus according to the present embodiment will be described with reference to FIGS. 1 and 2. FIG. 1 is an external view of an X-ray CT apparatus 1, and FIG. 2 is a block diagram illustrating an overall structure. As illustrated in FIGS. 1 and 2, the X-ray CT apparatus 1 includes a gantry 100, a bed 105, and an operation unit 120.

The gantry 100 includes a turn plate 102, a gantry control device 108, a bed control device 109, and an X-ray control device 110.

Note that a three-dimensional coordinate system illustrated in FIG. 1 illustrates an example of definition of directions in the X-ray CT apparatus 1. The X axis, the Y axis, and the Z axis of the three-dimensional coordinate system are examples and are not limited thereto. In the following description, the X axis, the Y axis, and the Z axis in the X-ray CT apparatus 1 are defined in the same direction in any drawing. The Z-axis direction is a body axis direction of a subject. The Y-axis direction is an up-down direction of the subject, and is a direction parallel to a direction of gravitational force. The X-axis direction is a left-right direction of the subject, and is a horizontal direction orthogonal to the direction of gravitational force.

The turn plate 102 includes a rotation frame 40 including an opening portion 104, and an X-ray tube 51, a collimator 103, and an X-ray detection unit 56 that are mounted on the rotation frame 40. A subject 2 placed on the bed 105 enters the opening portion 104 of the rotation frame 40. The turn plate 102 is rotated around the periphery of the subject 2. The turn plate 102 has a tilt mechanism that rotates the turn plate 102 in a state of being inclined with respect to a top plate principal plane of the bed 105 in the rotation axis direction.

Note that the gantry 100 accommodates the turn plate 102 and the like in a housing (gantry cover) 101 as illustrated in FIG. 1.

The X-ray tube 51 is a device that irradiates the subject 2 placed on the bed 105 with X-rays. The collimator 103 is a device that limits an irradiation range of X-rays emitted from the X-ray tube 51. The X-ray detection unit 56 includes an X-ray detector 256a and a data collection device 256b. The X-ray detector 256a is a device that is disposed to face the X-ray tube 51 on the rotation frame 40 and measures a spatial distribution of transmitted X-rays by detecting the X-rays transmitted through the subject. The X-ray detector 256a is an X-ray detector in which a large number of X-ray detection elements are arranged in the rotation direction of the rotation frame 40 or are two-dimensionally arranged in the rotation direction and a rotation axis direction of the rotation frame 40. The data collection device 256b is a device that collects an X-ray amount detected by the X-ray detector 256a as digital data.

The gantry control device 108 is a device that controls the rotation of the turn plate 102, and a controller 55 that is a part thereof is mounted on the rotation frame 40. The bed control device 109 is a device that controls the up-down and front-rear movements of the bed 105.

The X-ray control device 110 is a device that controls the power input to the X-ray tube 51. High-voltage generation units 53 and 54 and a cooling device 52, which are parts of the X-ray control device 110, are mounted on the rotation frame 40.

The operation unit 120 includes an input device 121, an image calculation device 122, a display device 125, a storage device 123, and a system control device 124. The input device 121 is a device for inputting the name of the subject, an examination date and time, an imaging condition, and the like. Specifically, the input device 121 is a keyboard or a pointing device. The image calculation device 122 is a device that performs CT image reconstruction by performing calculation processing on measurement data sent from the data collection device 256b of the X-ray detection unit 56. The display device 125 is a device that displays a CT image created by the image calculation device 122, and is, for example, a liquid crystal display or the like. The storage device 123 is a device that stores data collected by the data collection device 256b and the image data of the CT image created by the image calculation device 122, and is, for example, a hard disk drive (HDD) or the like. The system control device 124 is a device that controls these devices, the gantry control device 108, the bed control device 109, and the X-ray control device 110.

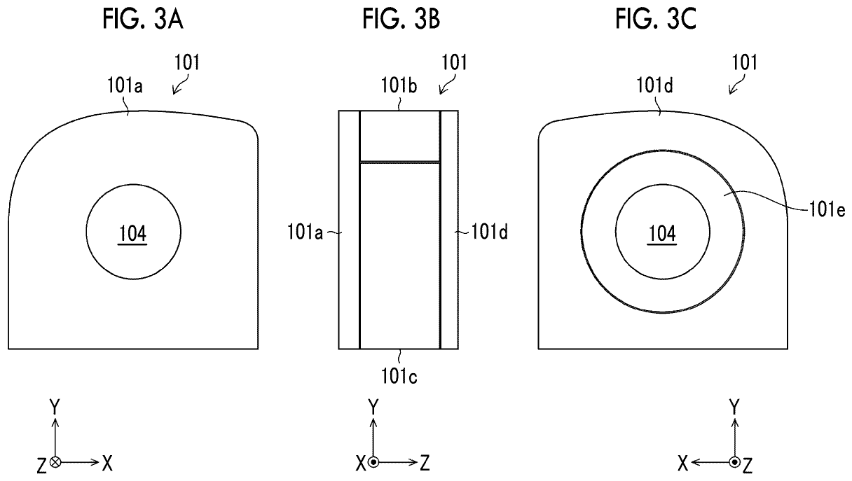

The structure of the gantry 100 will be described with reference to FIGS. 3 to 6.

FIGS. 3A to 3C are external views of the gantry 100, FIG. 3A is a front view, FIG. 3B is a side view, and FIG. 3C is a rear view. As illustrated in FIG. 3A to 3C, the gantry 100 accommodates the turn plate 102 and the like in the housing (gantry cover) 101. The gantry cover 101 is composed of a plurality of individual gantry covers. For example, the plurality of individual gantry covers include a front cover 101a, a top cover 101b, a side cover 101c, a first rear cover 101d, and a second rear cover 101e. By configuring the gantry cover 101 with the plurality of individual gantry covers, the assemblability and workability of the turn plate 102 and the like can be improved. The front cover 101a is constructed in one piece. The front cover 101a can be detached to expose the turn plate 102 and the like.

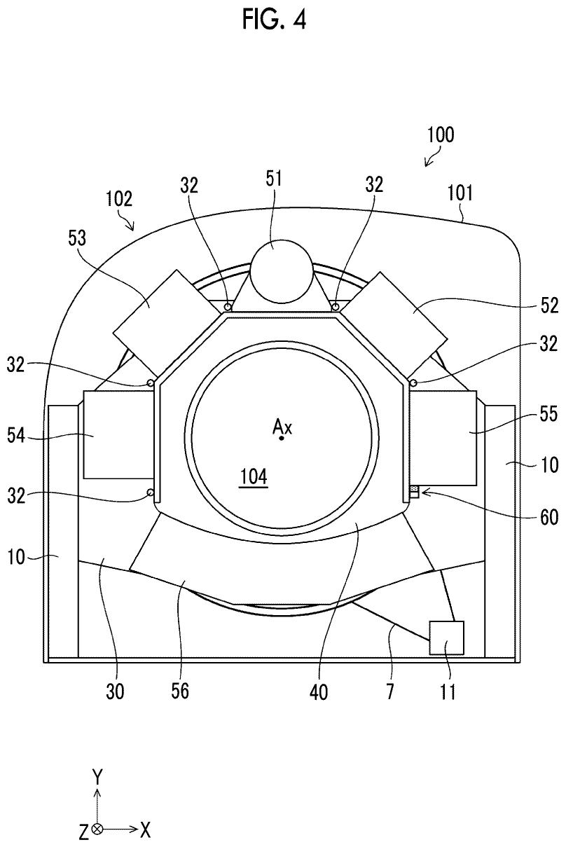

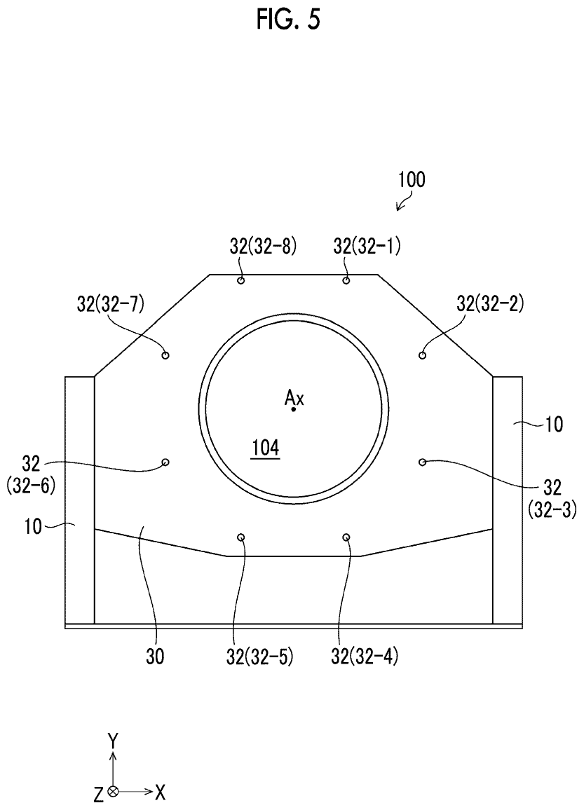

FIG. 4 is a front view of the turn plate 102 with the front cover 101a removed. FIG. 5 is a front view of a state where the rotation frame 40 is detached from the turn plate 102 of FIG. 4.

The gantry 100 includes the gantry cover 101 and the turn plate 102 (refer to FIG. 2 and the like) disposed inside the gantry cover 101. The turn plate 102 includes the rotation frame 40 in which the opening portion 104 is provided, a fixing frame 30 that supports the rotation frame 40 from the rear surface side via a bearing (not illustrated), and a stand 10 that supports the fixing frame 30.

The rotation frame 40 of the turn plate 102 is rotatably supported by the fixing frame 30 about a rotation axis Ax. The rotation frame 40 is connected to the large pulley (not illustrated) via a rotation portion side (inner ring) of the bearing. The large pulley is disposed on the rear surface side of the rotation frame 40. A driving belt 7 is wound around the outer periphery of the large pulley. A motor 11 is fixed at a position below the gantry 100 illustrated in FIG. 4 and close to the right side stand 10. A small pulley (not illustrated) is connected to the rotation axis (not illustrated) of the motor 11. The driving belt 7 is also wound around the small pulley.

The motor 11 rotationally drives the small pulley to drive the driving belt 7. The large pulley is rotated by driving the driving belt 7, and the rotation side (inner ring) of the bearing is rotated. In a case where the large pulley rotates the bearing, the rotation frame 40 is rotationally driven about the rotation axis Ax.

The rotation frame 40 has a substantially octagonal shape in a front view. The X-ray tube 51, the X-ray detection unit 56, the cooling device 52, the controller 55, the high-voltage generation units 54 and 53, and the like are mounted on the rotation frame 40. That is, the rotation frame 40 is provided with a plurality of components including the X-ray tube 51 and the X-ray detection unit 56. In FIG. 4, the plurality of components are fixed at positions corresponding to the sides of the rotation frame 40 having a substantially octagonal shape by bolts. The X-ray tube 51 is an example of an X-ray source according to the embodiment of the present invention. The X-ray detector 256a is an example of an X-ray detector according to the embodiment of the present invention. The X-ray tube 51, the X-ray detection unit 56, the cooling device 52, the controller 55, and the high-voltage generation units 54 and 53 are examples of the plurality of components according to the embodiment of the present invention. One lock mechanism 60 is disposed in the rotation frame 40.

As illustrated in FIG. 5, the fixing frame 30 is disposed on the rear surface side of the rotation frame 40. The fixing frame 30 has, for example, a substantially octagonal shape in a front view. An outer ring (not illustrated) that is a fixed portion side of the bearing is disposed on the fixing frame 30 to surround the opening portion 104. The fixing frame 30 is an example of the support frame according to the embodiment of the present invention. The rotation frame 40 is an example of the rotation frame according to the embodiment of the present invention.

One or more fixing holes 32, specifically, eight fixing holes 32 are formed in the fixing frame 30.

The imaging operation of the X-ray CT apparatus 1 will be briefly described. The imaging condition, particularly, an X-ray tube voltage, an X-ray tube current, and the like are input from the input device 121. The rotation frame 40 is rotated around the subject 2. The X-ray control device 110 controls the power input to the X-ray tube 51 on the basis of the imaging condition. The X-ray tube 51 irradiates the subject with X-rays according to the imaging conditions. The X-ray detection unit 56 detects the X-rays emitted from the X-ray tube 51 and transmitted through the subject by a plurality of X-ray detection elements of the X-ray detector 256a. The distribution of the transmitted X-rays is measured from a detection result. A rotation speed, an angle (tilt) of the rotation axis, and the like of the turn plate 102 are set to the imaging condition input from the input device 121 by the gantry control device 108. The rotation frame 40 is rotated and stopped by rotationally driving the driving belt 7 by the motor 11 and stopping the driving belt 7 by a brake (not illustrated). The gantry control device 108 controls the operation of the motor 11 and the operation of the brake. The bed 105 is controlled by the bed control device 109, and is operated on the basis of the imaging condition input from the input device 121, particularly, a helical pitch and the like.

The X-ray irradiation from the X-ray tube 51 and the measurement of the transmitted X-ray distribution by the X-ray detection unit 56 are repeated together with the rotation of the rotation frame 40. Accordingly, projection data at various angles is acquired by the data collection device 256b. The acquired projection data at various angles is transmitted to the image calculation device 122. The image calculation device 122 reconstructs the CT image by performing back-projection processing on the transmitted projection data at various angles. The CT image obtained by the reconstruction is displayed on the display device 125.

In a case of performing maintenance such as replacement of the X-ray tube 51 or the X-ray detection unit 56, the power of a rotation drive unit such as the motor 11 is turned off, and then the gantry cover 101 is removed. The rotation of the rotation frame 40 is regulated by the lock mechanism 60 to be in a locked state. The X-ray tube 51 and the like are detached from the rotation frame 40 in the locked state and are replaced or the like. In a case where the maintenance is completed, the lock mechanism 60 sets the rotation frame 40 to the unlocked state where the rotation frame 40 is rotatable, and the X-ray CT apparatus 1 starts the normal imaging operation.

### Lock Mechanism

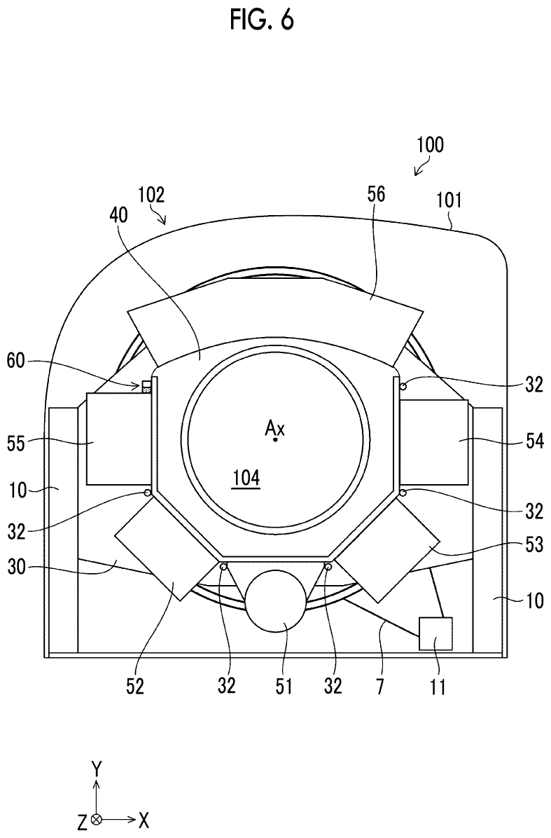

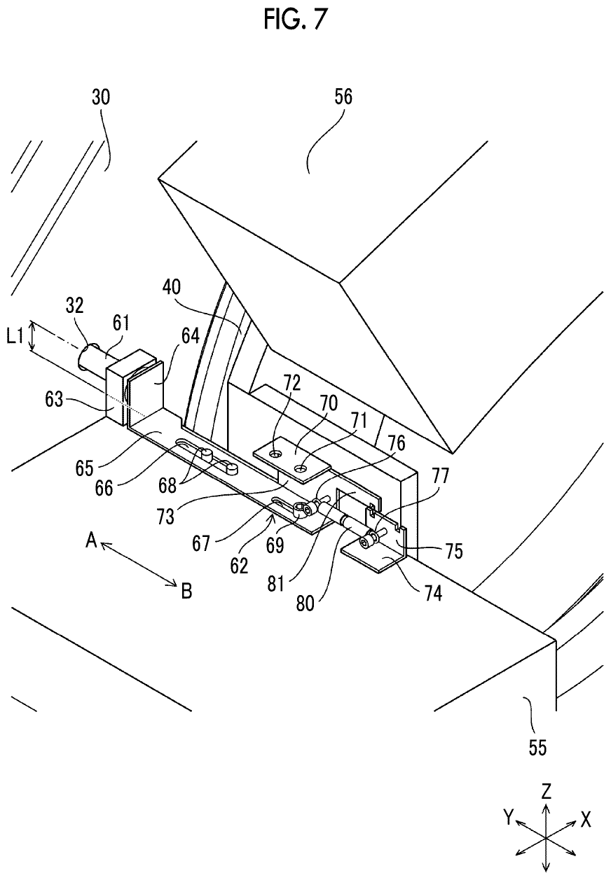

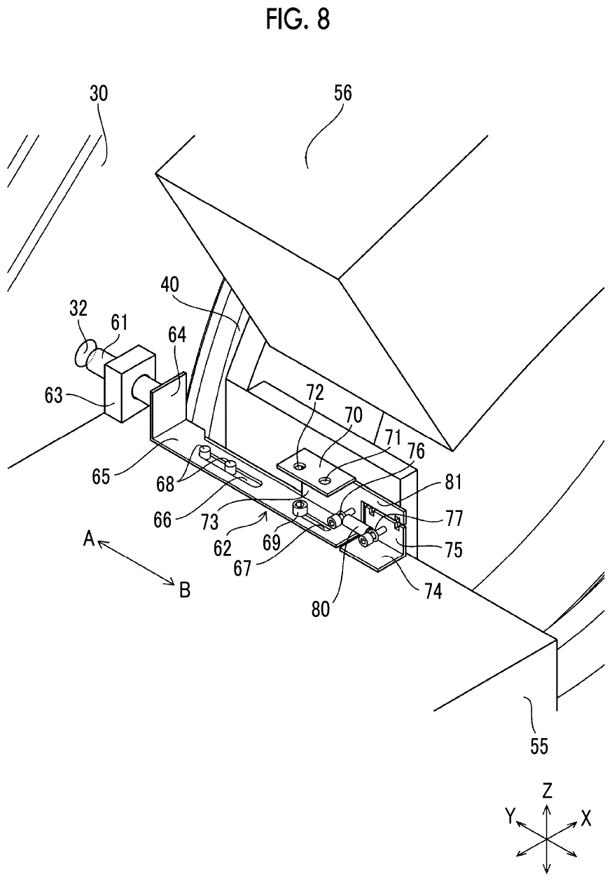

Next, the structures of the lock mechanism 60 and the fixing holes 32 that switch between the locked state and the unlocked state of the rotation frame 40 will be described. FIG. 6 is a diagram illustrating a state where the rotation frame 40 is locked by the lock mechanism 60. FIGS. 7 and 8 are diagrams for describing the operation of the lock mechanism 60.

In the state where the rotation frame 40 illustrated in FIG. 6 is locked, the X-ray tube 51, which is a target component for the maintenance, is positioned below the rotation axis Ax. In the maintenance, the target component is positioned below the rotation axis Ax, so that the work such as detachment and replacement of the target component is facilitated. In FIG. 6, the lock mechanism 60 is moved to a position facing the fixing hole 32 (position of 32-7: refer to FIG. 5) on the left side and second from the top such that the X-ray tube 51 is positioned below the rotation axis Ax.

FIG. 7 illustrates the locked state in which the rotation of the rotation frame 40 is restricted, and FIG. 8 illustrates the unlocked state in which the rotation frame 40 is rotatable. In the lock mechanism 60 of the embodiment, the rotation frame 40 is locked by causing a fixing shaft 61 of the lock mechanism 60 to engage with the fixing hole 32, and the rotation frame 40 is in the locked state in which the rotation is restricted. In addition, in a case where the engagement between the fixing shaft 61 of the lock mechanism 60 and the fixing holes 32 is released, the rotation frame 40 is in the unlocked state in which the rotation frame 40 is rotatable.

The fixing shaft 61 is an example of the fixing shaft of the embodiment of the present invention. The fixing hole 32 is an example of a lock fixing unit of the embodiment of the present invention. The shape of the lock fixing unit is not limited to a groove, a recess, or the like as long as the lock fixing unit can engage with the fixing shaft 61.

As illustrated in FIGS. 7 and 8, the lock mechanism 60 includes the fixing shaft 61 and a support member 62 that movably supports the fixing shaft 61. The fixing shaft 61 is a columnar rod-like member. An outer diameter of the fixing shaft 61 is a size so that the fixing shaft 61 can be inserted into the fixing hole 32. The fixing shaft 61 is inserted into the fixing hole 32, and the fixing shaft 61 and the fixing hole 32 engage with each other. The fixing shaft 61 is disposed in a direction along the Z-axis direction.

A side of the fixing shaft 61 that does not engage with the fixing hole 32 is movably inserted into a block body 63. The block body 63 supports the fixing shaft 61. The block body 63 has a substantially rectangular parallelepiped shape. The block body 63 is attached to the controller 55 in a state where the block body 63 cannot be moved with respect to the controller 55.

The support member 62 is movably attached to the controller 55. As described above, the controller 55 is one of the components mounted on the rotation frame 40. The support member 62 has a shape in which a plate-shaped member is bent. The support member 62 includes a plurality of plane portions. The support member 62 includes a connection surface 64 connected to the fixing shaft 61 and the block body 63. The connection surface 64 is provided to face the fixing frame 30.

The support member 62 includes a moving surface 65 which is movably attached to the controller 55. The moving surface 65 extends along the Z-axis direction. The connection surface 64 and the moving surface 65 are in a positional relationship substantially orthogonal to each other. A first long hole 66 and a second long hole 67 are formed in the moving surface 65. The first long hole 66 and the second long hole 67 extend along the Z-axis direction.

Two guide pins 68 are inserted into the first long hole 66. The moving surface 65 can smoothly move in an arrow A direction or an arrow B direction of the arrow B along the first long hole 66 by the guide pins 68. The A direction is a direction in which the rotation frame 40 is in the locked state, and the B direction is a direction in which the rotation frame 40 is in the unlocked state. A fixing screw 69 is inserted into the second long hole 67. The moving surface 65 can be fixed to the controller 55 in a state where the moving surface 65 cannot be moved, by fastening the fixing screw 69. The locked state and the unlocked state can be switched by changing the fastening position of the moving surface 65 with respect to the fixing screw 69. The fixing screw 69 is provided in a state where the fixing screw 69 cannot be moved with respect to the controller 55.

The support member 62 includes an access surface 70 at a position facing the second long hole 67. A first through-hole 71 and a second through-hole 72 are formed in the access surface 70. The access surface 70 is supported by a support surface 73 connected to the moving surface 65. The first through-hole 71 or the second through-hole 72 allows access to the fixing screw 69 only in a case where the first through-hole 71 or the second through-hole 72 coincides with an axial direction of the fixing screw 69. Therefore, the operator can tighten or loosen the fixing screw 69 with a tool via the first through-hole 71 or the second through-hole 72.

The support member 62 has a first fixing surface 74 on a side opposite to the connection surface 64 with respect to the moving surface 65. The first fixing surface 74 is fixed to the controller 55 in a state where the first fixing surface 74 cannot be moved. A second fixing surface 75 extending along the Z-axis direction is connected to the first fixing surface 74. The first fixing surface 74 and the second fixing surface 75 have a substantially L-shape as viewed from the Z-axis direction.

A first fixing pin 76 is provided on the support surface 73. The first fixing pin 76 is substantially orthogonal to the support surface 73. A second fixing pin 77 is provided on the second fixing surface 75. The second fixing pin 77 is substantially orthogonal to the second fixing surface 75. One end of a tension spring 80 is connected to the first fixing pin 76. The other end of the tension spring 80 is connected to the second fixing pin 77. The tension spring 80 biases the moving surface 65 in a direction (arrow B direction) of the first fixing surface 74. That is, the tension spring 80 biases the moving surface 65 in a direction in which the engagement between the fixing shaft 61 and the fixing hole 32 is released (to be in the unlocked state). The tension spring 80 is an example of an elastic member according to the embodiment of the present invention.

Note that, instead of the tension spring 80, a compression spring can be used as the clastic member. In addition, the moving surface 65 may be moved by a screw instead of the tension spring 80. As long as the moving surface 65 can be moved to the lock position and the unlock position, a unit for moving the moving surface 65 is not particularly limited.

In FIG. 7, the moving surface 65 is moved to a position where the fixing shaft 61 and the fixing hole 32 engage with each other against a tension force of the tension spring 80, and is fixed to the controller 55 by the fixing screw 69. That is, the moving surface 65 (support member 62) is fixed at a position (lock position) at which the rotation frame 40 is in the locked state.

In FIG. 8, the moving surface 65 is moved to a position where the engagement between the fixing shaft 61 and the fixing hole 32 is released by the tension force of the tension spring 80, and is fixed to the controller 55 by the fixing screw 69. That is, the moving surface 65 (support member 62) is fixed at a position (unlock position) at which the rotation frame 40 is in the unlocked state. Since the tension spring 80 pulls the moving surface 65 in the direction of the unlock position, the fixing shaft 61 can be prevented from engaging with the fixing hole 32 during the operation of the X-ray CT apparatus 1.

As illustrated in FIGS. 7 and 8, the longitudinal axis of the fixing shaft 61 and the longitudinal axis of the moving surface 65 of the support member 62 are offset from each other by a distance L1 as viewed from the Z-axis direction (movement direction of the fixing shaft 61 and the moving surface 65). Since the fixing shaft 61 is offset from the moving surface 65 (support member 62), the operator can easily check the positional relationship between the fixing shaft 61 and the fixing hole 32.

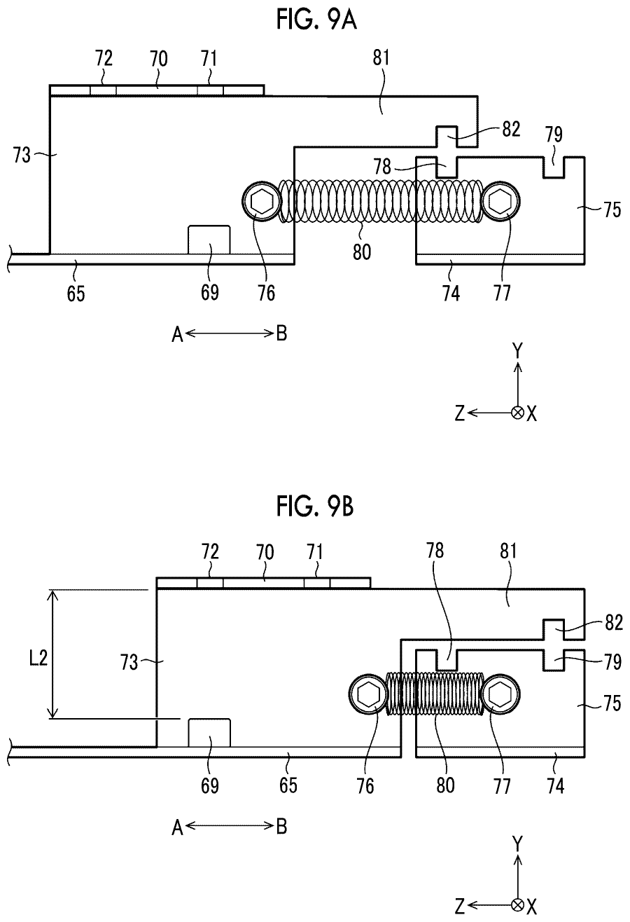

FIGS. 9A and 9B are diagrams for describing an indicator of the embodiment. FIG. 9A is a diagram illustrating the locked state, and FIG. 9B is a diagram illustrating the unlocked state.

The second fixing surface 75 is provided with a first notch 78 and a second notch 79 on a side opposite to the first fixing surface 74. The first notch 78 and the second notch 79 are spaced from each other in the Z-axis direction. The support surface 73 includes an indicator surface 81 that extends toward the second fixing surface 75. The indicator surface 81 is provided with a third notch 82 facing the second fixing surface 75. The combination of the first notch 78 and the second notch 79 and the third notch 82 is an indicator indicating whether the support member 62 is at a position where the rotation frame 40 is in the locked state or at a position where the rotation frame 40 is in the unlocked state.

As illustrated in FIG. 9A, in the locked state, the first notch 78 and the third notch 82 are located at positions facing each other as viewed from the axial direction of the first fixing pin 76 and the second fixing pin 77. That is, in a case where the first notch 78 and the third notch 82 face each other, the operator can recognize that the moving surface 65 is at the lock position where the fixing shaft 61 and the fixing hole 32 engage with each other.

As illustrated in FIG. 9B, in the unlocked state, the second notch 79 and the third notch 82 are located at positions facing each other as viewed from the axial direction of the first fixing pin 76 and the second fixing pin 77. That is, in a case where the second notch 79 and the third notch 82 face each other, the operator can recognize that the moving surface 65 is at the unlock position where the engagement between the fixing shaft 61 and the fixing hole 32 is released.

The first notch 78, the second notch 79, and the third notch 82 are examples of the indicator of the embodiment of the present invention. In the embodiment of FIGS. 9A and 9B, a case where notches are used as the indicator has been described. However, the present invention is not limited to the notch as long as the notch is an indicator indicating whether the rotation frame 40 is in the locked state or the unlocked state. For example, the indicator may be a groove, a protrusion, or a printed mark provided on the second fixing surface 75 and the indicator surface 81.

In addition, as illustrated in FIG. 9A, in a case where the moving surface 65 (support member 62) is positioned at the lock position, since the first through-hole 71 of the access surface 70 and the axial direction of the fixing screw 69 coincide with each other, the operator can access the fixing screw 69 via the first through-hole 71.

In addition, as illustrated in FIG. 9B, in a case where the moving surface 65 (support member 62) is positioned at the unlock position, since the second through-hole 72 of the access surface 70 and the axial direction of the fixing screw 69 coincide with each other, the operator can access the fixing screw 69 via the second through-hole 72. That is, the operator can access the fixing screw 69 via the first through-hole 71 or the second through-hole 72 of the access surface 70 only in a case where the moving surface 65 is positioned at the lock position or the unlock position. The moving surface 65 (support member 62) can be fixed only at the lock position or the unlock position.

A distance L2 between a screw head of the fixing screw 69 in a fastened state and the access surface 70 is shorter than a screw length of the fixing screw 69. In addition, the opening diameters of the first through-hole 71 and the second through-hole 72 are smaller than the diameter of the screw head of the fixing screw 69. The access surface 70 on which the first through-hole 71 and the second through-hole 72 are formed functions to prevent the fixing screw 69 from falling out. In a case of maintenance, the lock mechanism 60 is moved to a position facing any one of the eight fixing holes 32, and the fixing shaft 61 and the fixing hole 32 engage with each other. In this case, even in a case where the lock mechanism 60 is moved to the position of any of the fixing holes 32, the access surface 70 prevents the falling of the fixing screw 69 during the work. As a result, the workability of maintenance can be improved.

Next, a relationship between a combination of the locked states in which one of the eight fixing holes 32 and the fixing shaft 61 engage with each other and the maintenance component positioned at the work position will be described. In the following description, it is assumed that the work position is below the rotation axis Ax. Table 1 displays combinations of the position of the fixing hole 32 with which the fixing shaft 61 engages and the component name that is positioned at the work position, on the basis of the structure of the gantry 100 illustrated in FIGS. 4 to 6.

In Table 1, the positions of the fixing holes 32 are defined as 32-1 to 32-8 clockwise from the upper right position on the basis of FIG. 5.

TABLE 1

| Position of fixing hole | Component name |
|---|---|
| 32-1 | High-voltage generation unit |
| 32-2 | X-ray detection unit |
| 32-3 | X-ray detection unit |
| 32-4 | X-ray detection unit |
| 32-5 | Controller |
| 32-6 | Cooling device |
| 32-7 | X-ray tube |
| 32-8 | High-voltage generation unit |

In Table 1, for example, (position of fixing hole: 32-1, component name: high-voltage generation unit) indicates that the high-voltage generation unit 54 is positioned at the work position in a case where the fixing shaft 61 of the lock mechanism 60 engages with the fixing hole 32 at the position of 32-1. FIG. 6 corresponds to a case of (position of fixing hole: 32-7, component name: X-ray tube), and illustrates that the X-ray tube 51 is positioned at the work position in a case where the fixing shaft 61 of the lock mechanism 60 engages with the fixing hole 32 at the position of 32-7.

The lock mechanism 60 is preferably provided at a position where a condition is satisfied in which the fixing shaft 61 can engage with the fixing hole 32 above the rotation axis Ax and the component having a high replacement frequency can be positioned below the rotation axis Ax. The component having a high replacement frequency is the X-ray tube 51, and the fixing hole 32 positioned above the rotation axis Ax is, for example, the fixing hole 32 positioned at the position of 32-1, 32-2, 32-7, or 32-8.

Specifically, as illustrated in FIG. 6, the lock mechanism 60 is provided at a position adjacent to the controller 55 in the clockwise direction, in a front view. By providing the lock mechanism 60 at this position, the fixing shaft 61 can engage with the fixing hole 32 at the position of 32-7, and the X-ray tube 51 can be positioned at the work position.

In addition, unlike FIG. 4 or FIG. 6, the lock mechanism 60 may be provided at a position adjacent to the high-voltage generation unit 54 in the counterclockwise direction, in a front view. In this case, the fixing shaft 61 can engage with the fixing hole 32 at the position of 32-2, and the X-ray tube 51 is positioned at the work position. Note that, in a case where the lock mechanism 60 is provided at a position different from that in FIG. 4 or FIG. 6, the combination of the position of the fixing hole 32 and the component name is different from that in Table 1.

By causing the fixing shaft 61 to face the fixing hole 32 above the rotation axis Ax, the operator can cause the fixing shaft 61 to engage with the fixing hole 32 at the height of the line of sight. That is, the workability of maintenance for the operator can be improved. In particular, it is possible to reduce the burden of the engagement work for the component having a high frequency of maintenance. Note that the work position is described as being below the rotation axis Ax. However, depending on the devices, the unit may be positioned at a position (work position) above the rotation axis Ax and the maintenance work of the unit may be performed. That is, as long as the device can be positioned at a position desired by the operator, the work position may be above or below the rotation axis Ax.

In addition, it is preferable to improve safety in a case of maintenance. For example, in a case where the fixing shaft 61 and the fixing hole 32 engage with each other and the rotation frame 40 is in the locked state, it is preferable to turn off the power of the motor 11. Accordingly, in a case where the fixing shaft 61 and the fixing hole 32 engage with each other, since the power of the motor 11 is turned off, the rotation driving of the rotation frame 40 can be avoided. The engagement state between the fixing shaft 61 and the fixing hole 32 can be detected by providing a sensor (not illustrated). A detection signal from the sensor is transmitted to the gantry control device 108, and the gantry control device 108 turns off the power of the motor 11. In a case where the rotation frame 40 is in the locked state, the rotation frame 40 is not rotationally driven, and thus, the safety of the operator can be secured. The gantry control device 108 is an example of a processor according to the embodiment of the present invention.

Note that, in a case where the power of the motor 11 is once turned off, the power of the motor 11 is maintained in the off state even in a case where the engagement between the fixing shaft 61 and the fixing hole 32 is released. Sudden rotational drive by the rotation frame 40 can be avoided. In a case where the power is turned on, the operator manually turns on the power of the motor 11.

Even in a case where the rotation frame 40 is rotationally driven at the maximum output of the motor 11 in a state where the fixing shaft 61 and the fixing hole 32 engage with each other, the fixing shaft 61 has a strength to not be destroyed. The fixing shaft 61 can maintain the engagement state with the fixing hole 32, and the locked state of the rotation frame 40 can be maintained. Even in a case where the motor 11 rotationally drives the rotation frame 40, the fixing shaft 61 maintains the locked state of the rotation frame 40, and thus the safety of the operator can be secured.

In the embodiment, the plurality of fixing holes 32 and the one lock mechanism 60 have been described. However, the present invention is not limited to this, and may include one fixing hole 32 and one or more lock mechanisms 60. For example, eight lock mechanisms 60 may be provided on each side of the rotation frame 40 having a substantially octagonal shape. One fixing hole 32 can be provided above the rotation axis Ax. Therefore, even in a case where any replacement component is positioned at the work position, the operator can operate the lock mechanism 60 at the position of the line of sight. Further, it goes without saying that the present invention is not limited to the embodiments described above, and that various modifications are possible.

### EXPLANATION OF REFERENCES

30: fixing frame
32: fixing hole
40: rotation frame
55: controller
56: X-ray detection unit
60: lock mechanism
61: fixing shaft
62: support member
63: block body
64: connection surface
65: moving surface
66: first long hole
67: second long hole
68: guide pin
69: fixing screw
70: access surface
71: first through-hole
72: second through-hole
73: support surface
74: first fixing surface
75: second fixing surface
76: first fixing pin
77: second fixing pin
80: tension spring

What is claimed is:

1. An X-ray CT apparatus comprising:
a plurality of components including an X-ray source and an X-ray detector;
a rotation frame on which the plurality of components are mounted and which rotates; and
a support frame that rotatably supports the rotation frame,
wherein the support frame includes one or more lock mechanisms including a fixing shaft and a support member that movably supports the fixing shaft,
the support frame includes one or more lock fixing units engageable with the fixing shaft, and
the lock mechanism switches between a locked state in which rotation of the rotation frame is restricted and an unlocked state in which the rotation of the rotation frame is allowed.

2. The X-ray CT apparatus according to claim 1,
wherein in the locked state in which the fixing shaft of the lock mechanism and the lock fixing unit engage with each other, any one of the plurality of components is positioned at a position below a rotation axis of the rotation frame.

3. The X-ray CT apparatus according to claim 2,
wherein any one of the plurality of components is the X-ray source.

4. The X-ray CT apparatus according to claim 1,
wherein the one or more lock fixing units include a plurality of lock fixing units.

5. The X-ray CT apparatus according to claim 4,
wherein any component of the plurality of components is positioned at a predetermined position depending on a combination of locked states in which one of the plurality of lock fixing units and the fixing shaft of the lock mechanism engage with each other.

6. The X-ray CT apparatus according to claim 1,
wherein the one or more lock mechanisms include a plurality of lock mechanisms.

7. The X-ray CT apparatus according to claim 6,
wherein any component of the plurality of components is positioned at a predetermined position depending on a combination of locked states in which the fixing shaft of one lock mechanism of the plurality of lock mechanisms and the lock fixing unit engage with each other.

8. The X-ray CT apparatus according to claim 1,
wherein the fixing shaft is disposed to be offset from the support member.

9. The X-ray CT apparatus according to claim 1,
wherein the lock mechanism includes an indicator indicating whether the support member is at a position where the rotation frame is locked or at a position where the rotation frame is unlocked.

10. The X-ray CT apparatus according to claim 1,
wherein the lock mechanism includes an elastic member that biases the support member to a position where the rotation frame is in the unlocked state.

11. The X-ray CT apparatus according to claim 1, further comprising:
a motor that rotates the rotation frame; and
a processor that turns off power of the motor in a case where the rotation frame is in the locked state.

12. The X-ray CT apparatus according to claim 11,
wherein in a state where the fixing shaft engages with the lock fixing unit, the fixing shaft maintains the rotation frame in the locked state against a maximum output of the motor.
